(19) United States
(12) Patent Application Publication
(10) Pub. No.: US 2026/0308112 A1
(43) Pub. Date: Oct. 8, 2026

(54) SYNTHESIS OF N-LACTOYL-PHENYLALANINE (LAC-PHE) BY CONDENSATION OF A PHENYLALANINE ESTER AND A LACTIC ACID ESTER

(71) Applicant: KEMIN INDUSTRIES, INC., DES MOINES, IA (US)

(72) Inventors: Andrew RIDGWAY, West Des Moines, IA (US); Frederick KHACHIK, Urbandale, IA (US)

(73) Assignee: KEMIN INDUSTRIES, INC., DES MOINES, IA (US)

(21) Appl. No.: 19/641,469

(22) Filed: Apr. 7, 2026

Related U.S. Application Data
(60) Provisional application No. 63/784,614, filed on Apr. 7, 2025.

(51) Int. Cl. C07C 231/02; C07C 231/24

(52) U.S. Cl. CPC C07C 231/02; C07C 231/24

(57) ABSTRACT

The present invention relates to a novel two-step process for synthesis of (L,L)-Lac-Phe (major product) and (L,D)-Lac-Phe (minor product) from L-phenylalanine ester hydrochloride and L-lactic acid ester under mild conditions. In the first step, a reaction mixture comprising L-phenylalanine ester hydrochloride is deprotonated with tertiary amine at ambient temperature using L-lactic acid ester as solvent. The resulting mixture is then treated with an acid and heated to yield (L,L)-Lac-Phe ester as the major product and (L,D)-Lac-Phe ester as the minor product. In the second step, (L,L)- and (L,D)-Lac-Phe esters are subjected to alkaline hydrolysis to yield (L,L)-Lac-Phe (major product) and (L,D)-Lac-Phe (minor product). Another aspect relates to a similar process, wherein D-phenylalanine ester hydrochloride is condensed with L-lactic acid ester to obtain (L,D)-Lac-Phe ester as the major product and (L,L)-lac-Phe ester as the minor product and saponified to yield (L,D)-Lac-Phe as the major product and (L,L)-Lac-Phe as the minor product.

### CROSS-REFERENCE TO RELATED APPLICATIONS

The present application claims the benefit of priority to U.S. Provisional Patent Application No. 63/784,614, filed Apr. 7, 2025, entitled “SYNTHESIS OF N-LACTOYL-PHENYLALANINE (LAC-PHE) BY CONDENSATION OF A PHENYLALANINE ESTER AND A LACTIC ACID ESTER,” the entire disclosure of which is hereby incorporated by reference in its entirety.

### TECHNICAL FIELD

The present invention relates to a novel process for synthesis of N-lactoyl-phenylalanine (Lac-Phe) by thermal condensation of commercially available phenylalanine methyl ester hydrochloride with ethyl L-lactate followed by alkaline hydrolysis. In another aspect, the present invention employs commercially available L-phenylalanine methyl ester hydrochloride and ethyl L-lactate as starting materials that in two steps afford Lac-Phe in good to moderate yields (i.e., greater than 50%) under mild reaction conditions. In the first step, thermal condensation of deprotonated L-phenylalanine methyl ester hydrochloride and ethyl L-lactate affords N-lactoyl-phenylalanine methyl ester (Lac-Phe methyl ester) that in the second step is saponified to Lac-Phe, quantitatively.

### BACKGROUND OF INVENTION

The present invention relates to a novel process for synthesis of N-lactoyl-phenylalanine (Lac-Phe) that has been accomplished in two steps under mild reaction conditions. The first step of this process involves deprotonation of L-phenylalanine methyl ester hydrochloride with triethylamine that, without isolation is condensed with ethyl L-lactate at temperatures in the range of about 45-60° C. to afford (L,L)-Lac-Phe methyl ester. In the second step of this process, (L,L)-Lac-Phe methyl ester is subjected to alkaline hydrolysis to yield (L,L)-Lac-Phe almost quantitatively. This process has also been applied to the synthesis of (L,D)-Lac-Phe that has been prepared by thermal condensation of deprotonated D-phenylalanine methyl ester hydrochloride and ethyl L-lactate followed by alkaline hydrolysis.

Recent advances in the weight management space have identified (L,L)-N-Lactoyl-Phenylalanine [(L,L)-Lac-Phe] as a potential new satiety ingredient (Li, Veronica L.; Yang He; Kévin Contrepois, et al. 2022). (L,L)-Lac-Phe is a naturally occurring, blood borne metabolite that is produced by macrophage cells in the vicinity of the intestinal lumen. Presence of Lac-Phe in the blood serum has been associated with lower feed intake and weight loss in obese mice. The effect is thought to mimic the body's appetite suppressing response to high intensity exercise (Xiao, Shuke, Veronica L. Li, Xuchao Lyu, et al. 2023). Lac-Phe is available in small quantities (mg) and a high cost as an analytical standard and has been synthesized by condensation of phenylalanine with calcium lactate at high temperature (Wu, Jing; Jingrong Gao; Junjie Lin, et al. 2022). However, this reported synthesis lacks clarity and sufficient detail regarding isolated yield to determine its commercial viability. Notably, the stereochemistry of Lac-Phe with two stereocenters was not addressed and the isolated yield was not reported. Instead, the authors reported their yield based on HPLC data and Lac-Phe was isolated in small quantities by preparative HPLC.

The objective of this invention was to develop a novel methodology for synthesis of Lac-Phe using green chemistry. Lac-Phe has been previously synthesized using traditional organic chemistry reagents such as hexafluorophosphate benzotriazole tetramethyl uronium (HBTU) and lithium hydroxide in dichloromethane (Li, Veronica L.; Yang He; Kévin Contrepois et al. 2022). However, an approach to the synthesis of Lac-Phe that could utilize ‘green’ reagents and class II or III solvents would have significant processing and regulatory advantages. Our numerous attempts to synthesize Lac-Phe by condensation of phenylalanine and lactic acid or its salts according to published procedures were unsuccessful or in best cases afforded a very low yield of the desired product (Gooßen, Lukas; Dominik Ohlmann; Paul Lange. 2009). Therefore, we envisioned a different synthetic methodology by employing a commercially available and protected L-phenylalanine methyl ester hydrochloride and ethyl L-Lactate as starting materials. Although the use of a protected amino acids has been extensively applied to synthesis of many peptides (Taussat, Armand; Renata Marcia de Figueiredo; Jean-Marc Campagne. 2023), there is no literature report on the synthesis of Lac-Phe according to our synthetic strategy.

### SUMMARY OF THE INVENTION

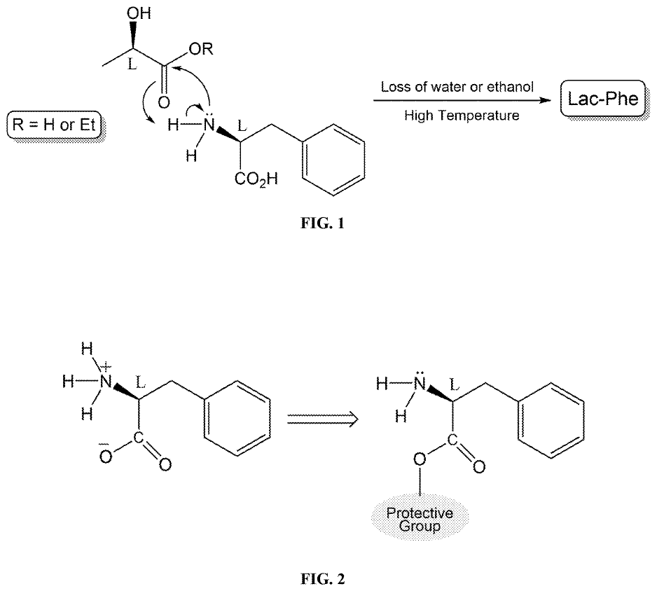

In a typical peptide synthesis, an amino acid is condensed with an organic acid, its salt or its ester to form a peptide bond. As shown in FIG. 1, this reaction is promoted by the loss of water or an alcohol. As a result, a dehydrant, a high temperature or both are needed to promote this reaction. At high temperature, chiral amino acid can also undergo epimerization that compromises the stereochemical integrity of the resulting peptide.

As mentioned previously, Wu et al. (2022) condensed phenylalanine with calcium lactate to prepare Lac-Phe at 100° C. for 3 h but did not address the stereochemistry of their product and they only isolated small quantities of Lac-Phe by preparative HPLC. Our numerous attempts to follow this reported procedure under a wide range of conditions and temperatures was unsuccessful and we were only able to obtain 14% yield of Lac-Phe that consisted of a mixture of (L,L)- and (L,D)-isomers.

The unprotected phenylalanine exists as a zwitterion ion and exhibits poor reactivity in condensation reactions, particularly at moderate temperatures. Therefore, we anticipated that the protected L-phenylalanine methyl ester would have a significantly greater reactivity in condensation with ethyl L-lactate (FIG. 2). In addition, at the conclusion of our synthesis, the removal of the ester protecting group would be simply accomplished by alkaline hydrolysis.

The present invention relates generally to a process for synthesis of N-lactoyl-phenylalanine (Lac-Phe) in two steps from commercially available L-Phenylalanine methyl ester hydrochloride and ethyl L-lactate.

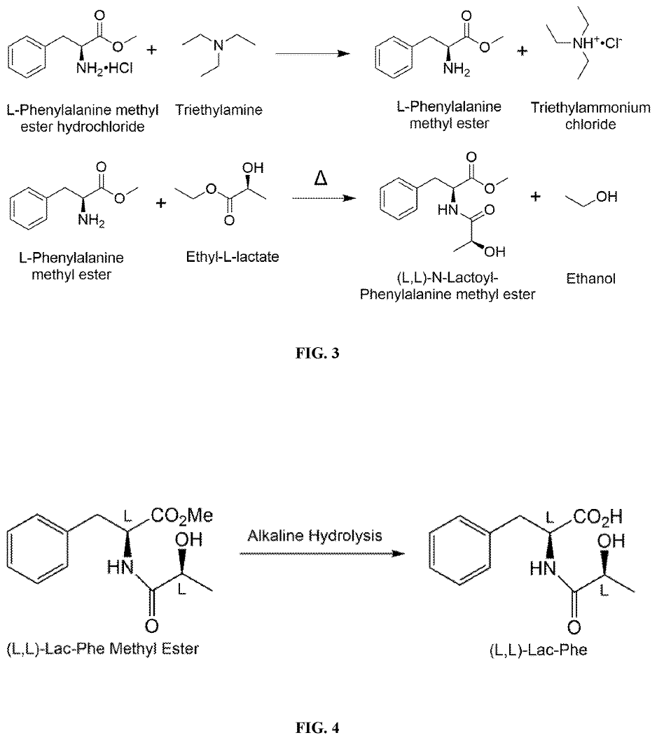

The inventors have surprisingly discovered a novel process for condensation of ethyl L-lactate with protected L-phenylalanine methyl ester after deprotection of its hydrochloride salt to afford N-lactoyl-phenylalanine methyl ester (Lac-Phe methyl ester) (FIG. 3).

The inventors have surprisingly been able to conduct this condensation under mild reaction conditions to obtain (L,L)-Lac-Phe methyl ester as the major product and (L,D)-Lac-Phe methyl ester as the minor product. The mild condensation temperature and reaction conditions solve the technical challenges in synthesis of Lac-Phe that is accompanied by significant loss of stereochemistry due to epimerization under harsh reaction conditions. In certain embodiments, “mild conditions” includes temperatures in the range of about 40 to 90° C. In certain embodiments, “harsh conditions” includes temperatures of about 100° C. or greater.

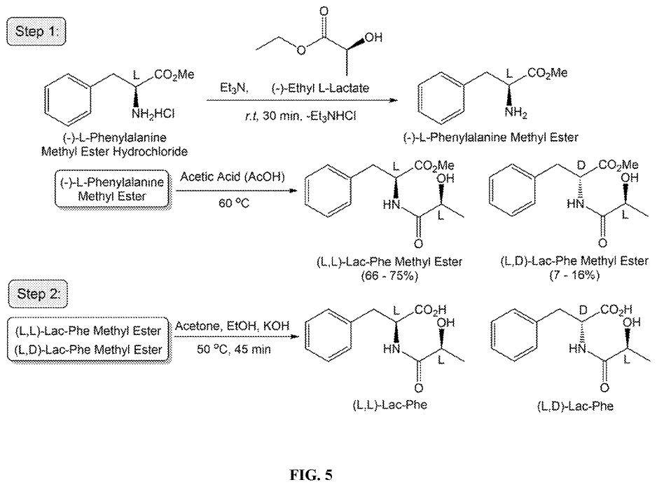

In the following step, the resulting Lac-Phe methyl ester is subjected to alkaline hydrolysis to afford Lac-Phe almost quantitatively (FIG. 4).

More specifically, in at least one embodiment, the reaction mixture comprising of commercially available L-phenylalanine methyl ester hydrochloride (1 eq.) in ethyl L-lactate (4 eq.) that is also used as solvent, is deprotonated with triethylamine (1 eq.) at ambient temperature. The resulting mixture is then treated with acetic acid (0.1-1 eq., preferably 0.5 eq.)⁶ and heated at temperatures in the range of 45-95° C., preferably 50-60° C. for 24-72 h to yield (L,L)-Lac-Phe methyl ester as the major product and (L,D)-Lac-Phe methyl ester as the minor product. In certain embodiments, the condensation of L-phenylalanine with ethyl L-lactate in the temperature range of 50-60° C. affords 66-75% yield of (L,L)-Lac-Phe methyl ester (major product) and (L,D)-Lac-Phe methyl ester (minor product) within 54-66 h in diastereomeric excess of 65-82%.

In certain embodiments, the product of condensation is filtered to remove triethylamine hydrochloride using acetone and the filtrate is evaporated under reduced pressure (350-150 torr) at 40-45° C. to afford crude condensation products. In one embodiment, the crude condensation products consist of a mixture of (L,L)-Lac-Phe methyl ester (66-75%) and (L,D)-Lac-Phe methyl ester (12-16%) in diastereomeric excess of 65-82%.

In certain embodiments, the crude condensation products are subjected to column chromatography on silica gel using hexane and acetone to isolate and separate (L,L)-Lac-Phe methyl ester from (L,D)-Lac-Phe methyl ester.

In certain embodiments, the crude mixture of (L,L)-Lac-Phe methyl ester and (L,D)-Lac-Phe methyl ester prior to purification is saponified with 50% aqueous solution of potassium hydroxide (KOH) at 50-60° C. to yield (L,L)-Lac-Phe and (L,D)-Lac-Phe, respectively. In another embodiment, acetone is optionally used as a co-solvent with 50% aqueous solution of KOH (wt.:wt.) in ethanol to saponify the crude mixture of (L,L)- and (L,D)-Lac-Phe methyl ester to (L,L)-Lac-Phe and (L,D)-Lac-Phe, respectively.

In certain embodiments, the crude saponified mixture of (L,L)-Lac-Phe and (L,D)-Lac-Phe is treated with a 50% solution of acetic acid-water until the solution is acidic (pH=4-5). Acetone is evaporated under reduced pressure at 40° C. and the crude product is partitioned into ethyl acetate and 5% solution of sodium chloride in water. The organic layer is washed with water and ethyl acetate is evaporated under reduced pressure to afford a crude saponified mixture of (L,L)- and (L,D)-Lac-Phe as a dark yellow oil.

In certain embodiments, the crude product of saponification is subjected to column chromatography on silica gel using hexane and ethyl acetate to isolate and purify a mixture of (L,L)-Lac-Phe (major product) and (L,D)-Lac-Phe (minor product).

In certain embodiments, the entire two-step process is carried out in a one-pot reaction without isolation of (L,L)- and (L,D)-Lac-Phe methyl esters to prepare a crude saponified mixture of (L,L)-Lac-Phe as the major product and (L,D)-Lac-Phe as the minor product (FIG. 5).

In certain embodiments, the crude product of one-pot reaction is subjected to column chromatography on silica gel using hexane and ethyl acetate to isolate and purify a mixture of (L,L)-Lac-Phe (major) and (L,D)-Lac-Phe (minor), respectively.

In another embodiment, commercially available D-phenylalanine methyl ester hydrochloride is similarly condensed with ethyl L-lactate at 45-60° C. for 24-72 h to yield (L,D)-Lac-Phe methyl ester as the major product and (L,L)-Lac-Phe methyl ester as the minor product after work up.

In certain embodiments, the above crude product is subjected to column chromatography on silica gel using hexane and acetone to isolate and separate (L,D)-phenylalanine methyl ester (major product) from (L,L)-phenylalanine methyl ester (minor product).

In certain embodiments, the crude product of one-pot reaction is subjected to column chromatography on silica gel using hexane and ethyl acetate to isolate and purify a mixture of (L,D)-Lac-Phe (major) and (L,L)-Lac-Phe (minor).

### DETAILED DESCRIPTION OF THE DRAWINGS

FIG. 1 illustrates synthesis of Lac-Phe by condensation of L-phenylalanine and L-lactic acid or its ethyl ester.

FIG. 2 illustrates zwitterion ion formation that is prevented by a protecting group.

FIG. 3 illustrates thermal condensation of deprotonated L-phenylalanine methyl ester hydrochloride with ethyl L-lactate.

FIG. 4 illustrates alkaline hydrolysis of (L,L)-Lac-Phe methyl ester to Lac-Phe.

FIG. 5 illustrates overall two-steps synthesis of (L,L)- and (L,D)-Lac-Phe.

### DETAILED DESCRIPTION OF THE INVENTION

The present invention relates to a novel two-step process for synthesis of (L,L)-Lac-Phe (major product) and (L,D)-Lac-Phe (minor product) from commercially available and protected L-phenylalanine methyl ester hydrochloride and ethyl L-lactate under mild conditions. In the first step, a reaction mixture comprising of commercially available L-phenylalanine methyl ester hydrochloride is deprotonated with triethylamine at ambient temperature using ethyl L-lactate as solvent. The resulting mixture is then treated with acetic acid and heated at an elevated temperature in the range of about 45-60° C. for 24-72 h to yield (L,L)-Lac-Phe methyl ester as the major product and (L,D)-Lac-Phe methyl ester as the minor product. In the second step, (L,L)- and (L,D)-Lac-Phe methyl esters are subjected to alkaline hydrolysis to yield (L,L)-Lac-Phe (major product) and (L,D)-Lac-Phe (minor product).

According to certain embodiments, L-phenylalanine methyl ester hydrochloride (1 eq.) in ethyl L-lactate (4 eq.) that is also used as solvent, is deprotonated with triethylamine (1 eq.) at ambient temperature within 30 min comprising: adding acetic acid (0.1-1 eq., preferably 0.5 eq.) and heating the mixture to a temperature, for instance to a range from about 45° C.-95° C.; preferably from about 45° C. to 60° C. for a duration of about 24-72 h, preferably 50-60 h to affect condensation of L-phenylalanine methyl ester with ethyl L-lactate to obtain a mixture of (L,L)-Lac Phe methyl ester (major product) and (L,D)-Lac-Phe methyl ester (minor product); diluting the crude product with ethyl acetate and filtering the mixture to remove triethylamine hydrochloride; evaporating acetone under reduced pressure (350-150 torr) at about 40-45° C. to obtain crude condensation product;

According to certain embodiments, the relative composition of (L,L)-Lac-Phe methyl ester in the crude product is in the range of about 66-75% and the relative composition of (L,D)-Lac-Phe methyl ester is 12-16% and only 9-12% of the starting material remains unreacted;

In certain embodiments, the diastereomeric excess of (L,L)-Lac-Phe methyl ester in the crude product is in the range of about 65-82%.

In certain embodiments, the condensation of L,L-phenylalanine methyl ester with ethyl L-lactate results in partial hydrolysis of (L,L)- and (L,D)-Lac-Phe methyl esters to (L,L)-Lac-Phe and (L,D)-Lac-Phe (2-8%), respectively.

Employing reversed phase HPLC, the inventors have followed the course of condensation of L-phenylalanine methyl ester with ethyl L-lactate under a wide range of conditions and have determined the relative composition of the crude products; the results are shown in Table 1.

In certain embodiments, the relative composition of (L,L)-Lac-Phe methyl ester was in the range of about 66-75% that was accompanied by its diastereomeric isomer, (L,D)-Lac-Phe methyl ester (7-16%). During prolonged reaction time, about 2-8% of (L,L)- and (L,D)-Lac-Phe methyl esters were hydrolyzed to (L,L)- and (L,D)-Lac-Phe, respectively.

TABLE 1
The relative composition of the products of condensation of (L,L)-
phenylalanine methyl ester with ethyl L-lactate determined by HPLC.

| | | | | | | L,L-Me- | L,D-Me- | |
|---|---|---|---|---|---|---|---|---|
| | Acetic | | Unreacted | | Lac- | Lac- | Lac- | L,L-Me- |
| | Acid | Temp | L-Phenylalanine | Time | Phe^{a} | Phe^{b} | Phe^{b} | Lac-Phe |
| Entry | eq | ° C. | Methyl Ester, % | hr | % | % | % | Excess^{c} % |
| 1 | 0.5 | 95 | 1 | 21 | 4 | 65 | 30 | 36 |
| 2 | 0.1 | 80 | 37 | 24 | 4 | 47 | 11 | 62 |
| 3 | 0.2 | 80 | 14 | 18 | 8 | 59 | 19 | 52 |
| 4 | 0.3 | 80 | 5 | 36 | 5 | 69 | 21 | 53 |
| 5 | 0.5 | 70 | 0 | 66 | 6 | 71 | 24 | 50 |
| 6 | 0.5 | 60 | 17 | 48 | 3 | 66 | 14 | 65 |
| 7 | 0.5 | 60 | 9 | 54 | 5 | 75 | 12 | 73 |
| 8 | 0.5 | 60 | 10 | 54 | 4 | 73 | 13 | 70 |
| 9 | 0.5 | 60 | 12 | 54 | 4 | 73 | 12 | 72 |
| 10 | 0.5 | 60 | 6 | 66 | 5 | 73 | 16 | 64 |
| 11 | 1 | 60 | 4 | 54 | 3 | 77 | 16 | 66 |
| 12 | 0.5 | 55 | 9 | 72 | 3 | 75 | 13 | 70 |
| 13 | 0.5 | 50 | 22 | 54 | 2 | 69 | 7 | 82 |
| 14 | 0.5 | 45 | 14 | 90 | 2 | 74 | 9 | 79 |
| 15 | 0.5 | 45 | 15 | 100 | 4 | 72 | 9 | 79 |

^{a}Lac-Phe was an isomeric mixture of (L,L)- and (L,D)-Lac-Phe,
^{b}Me-Lac-Phe refers to Lac-Phe methyl ester,
^{c}diastereomeric excess were calculated based on the relative composition of (L,L)-Lac-Phe methyl ester and (L,D)-Lac-Phe methyl ester. All reactions were performed with 1 equivalent of L-Phenylalanine methyl ester HCl and 4 equivalents Ethyl-L-lactate

In certain embodiments, the reaction time could be shortened at high temperatures of about 70-95° C., but this resulted in the formation of significant amounts of (L,D)-Lac-Phe methyl ester and a poor diastereomeric excess of the desired (L,L)-Lac-Phe methyl ester (Table 1, entries 1-5).

Therefore, in a preferred embodiment, the optimum temperature for the condensation reaction was found in the range of about 50-60° C.; this was based on HPLC analyses of the crude products of various reactions that were shown to afford (L,L)-Lac-Phe methyl ester in about 66-75% yield corresponding to about 65-82% diastereomeric excess (Table 1, entries 6-13).

In certain embodiments, by lowering the temperature of the condensation reactions to about 45° C., (L,L)-Lac-Phe methyl ester was obtained in an amount of about 70% or greater, for instance about 72-74% yield and 79% diastereomeric excess and was only accompanied by 9% of (L,D)-Lac-Phe methyl ester, while 14-15% of L-phenylalanine methyl ester remained unreacted (Table 1, entries 14-15). However, these conditions required prolonged reaction times of about 90 to 100 hours.

Diastereomeric excess was calculated by determining the relative composition of (L,L)- and (L,D)-Lac-Phe methyl esters and subtracting the relative composition of (L,D)-Lac-Phe methyl ester from the (L,L)-isomer. For example in entry 13 (Table 1), the relative composition of (L,L)-Lac-Phe methyl ester, with exclusion of other compounds, was 91% and that of (L,D)-Lac-Phe methyl ester was 9% that was subtracted from the relative composition of the (L,L)-isomer to arrive at 82% diastereomeric excess for (L,L)-Lac-Phe methyl ester.

In certain embodiments, the crude condensation products (10 g) were subjected to column chromatography on silica gel (175 g) using hexane (100% to 70%) and acetone (0% to 30%) to isolate and separate (L,L)-Lac-Phe methyl ester from (L,D)-Lac-Phe methyl ester. The structures of isolated (L,L)- and (L,D)-Lac-Phe methyl esters were confirmed by electrospray chemical ionization mass spectrometry (ESI-MS), ¹H- and ¹³C-nuclear magnetic resonance (¹H- and ¹³C-NMR) spectroscopy.

In certain embodiments, the crude mixture of (L,L)-Lac-Phe methyl ester and (L,D)-Lac-Phe methyl ester (10 g) prior to purification was saponified with 50% aqueous solution of potassium hydroxide (KOH, 8 g, wt.:wt) in ethanol (20 g) at 50-60° C. to yield (L,L)-Lac-Phe (major product) and (L,D)-Lac-Phe (minor product) within 30-45 min, respectively.

In another embodiment, acetone (30 g) is optionally used as a co-solvent with 50% aqueous solution of KOH (8 g, wt.:wt.) in ethanol (20 g) to saponify the crude mixture of (L,L)- and ((L,D)-Lac-Phe methyl ester (10 g) to (L,L)-Lac-Phe and (L,D)-Lac-Phe at 50-60° C. within 30-45 min, respectively.

In certain embodiments, the crude saponified mixture of (L,L)-Lac-Phe and (L,D)-Lac-Phe is treated with a 50% solution of acetic acid-water (v:v) until the solution is acidic (pH=4-5). Ethanol and acetone are evaporated under reduced pressure at about 40-45° C. and the crude product is partitioned into ethyl acetate and 5% solution of sodium chloride in water. The organic layer is washed with water and ethyl acetate is evaporated under reduced pressure to afford a crude saponified mixture of (L,L)- and (L,D)-Lac-Phe as a dark yellow oil.

In certain embodiments, the crude product of saponification (9 g) is subjected to column chromatography on silica gel (75 g) using hexane (100% to 30%) and ethyl acetate (0% to 70%) to isolate and purify a mixture of (L,L)-Lac-Phe (major product) and (L,D)-Lac-Phe (minor product). The structures of isolated (L,L)- and (L,D)-Lac-Phe were confirmed by ESI-MS) and ¹H- and ¹³C-NMR.

In certain embodiments, the entire two-step process is carried out in a one-pot reaction without isolation of (L,L)- and (L,D)-Lac-Phe methyl esters to prepare a crude saponified mixture of (L,L)-Lac-Phe as the major product and (L,D)-Lac-Phe as the minor product.

In certain embodiments, the crude product of one-pot reaction is subjected to column chromatography on silica gel using hexane and ethyl acetate to isolate and purify a mixture of (L,L)-Lac-Phe (major) and (L,D)-Lac-Phe (minor), respectively.

The following examples are offered to illustrate but not limit the invention. Thus, it is presented with the understanding that various formulation modifications as well as method of delivery modifications may be made and still are within the spirit of the invention.

### Example 1

### General Procedure for Condensation of L-Phenylalanine Methyl Ester Hydrochloride with Ethyl L-Lactate

All reactions were carried out on 3-5 g scale of L-phenylalanine methyl ester hydrochloride. In a typical reaction (3 g, 0.014 mol, 1 eq.) was transferred to a 100 mL round bottom flask with a stir bar and was suspended in ethyl L-lactate (6.57 g, 0.056 mol, 4 eq.). This amount was determined to be sufficient to maintain the flowability of the reaction mixture under various conditions outlined in Table 2. Triethylamine (1.42 g, 0.014 mol, 1 eq.) was added to the slurry and the mixture was stirred at ambient temperature (23-25° C.) for 30 minutes to allow deprotonation of L-phenylalanine methyl ester hydrochloride to L-phenylalanine methyl ester. An appropriate amount of glacial acetic acid (0.1 or 1 molar equivalent, Table 2) was added, the flask was equipped with a reflux condenser and placed in a preheated oil bath and stirred at temperatures and times described in Table 2. The course of the reaction was monitored by HPLC after 6 h and every 24 h thereafter. The reaction mixture was allowed to cool down to ambient temperature and was filtered using acetone (50 mL) to remove triethylamine hydrochloride. The filtrate was evaporated under reduced pressure (350-150 torr) at 40-45° C. to afford the crude products of each reaction (9-9.5 g).

TABLE 2
Reaction conditions for condensation of L-phenylalanine
methyl ester hydrochloride with ethyl L-lactate.

| | L-Phenyl alanine | Ethyl L- | | | |
|---|---|---|---|---|---|
| | Methyl Ester•HCl | Lactate | AcOH | Temp. | Time |
| Entry | eq. | eq. | eq. | 0° C. | h |
| 1 | 1 | 4 | 0.5 | 95 | 21 |
| 2 | 1 | 4 | 0.1 | 80 | 24 |
| 3 | 1 | 4 | 0.2 | 80 | 18 |
| 4 | 1 | 4 | 0.3 | 80 | 36 |
| 5 | 1 | 4 | 0.5 | 70 | 66 |
| 6 | 1 | 4 | 0.5 | 60 | 48 |
| 7 | 1 | 4 | 0.5 | 60 | 54 |
| 8 | 1 | 4 | 0.5 | 60 | 54 |
| 9 | 1 | 4 | 0.5 | 60 | 54 |
| 10 | 1 | 4 | 0.5 | 60 | 66 |
| 11 | 1 | 4 | 1 | 60 | 54 |
| 12 | 1 | 4 | 0.5 | 55 | 72 |
| 13 | 1 | 4 | 0.5 | 50 | 54 |
| 14 | 1 | 4 | 0.5 | 45 | 90 |
| 15 | 1 | 4 | 0.5 | 45 | 100 |

The condensation of D-phenylalanine methyl ester hydrochloride (1 eq.) with ethyl L-lactate (4 eq.) was carried out with 0.5 eq. of acetic acid at 55° C. for 72 h according to the above procedure.

### Example 2

### Isolation and Purification of (L,L)-Lac-Phe Methyl Ester and (L,D)-Lac-Phe Methyl Ester by Flash Column Chromatography

A flash column (length: 170 mm, internal diameter: 20 mL) was packed with silica gel (20 g) using hexane. The crude product (9 g) from condensation of L-phenylalanine methyl ester hydrochloride with ethyl L-lactate in acetone (10 mL) was loaded onto the column and the column was eluted with hexane and acetone as shown in Table 3. Based on HPLC analysis, fractions 7 and 8 were combined and evaporated to dryness for MS and NMR analysis; the combined fractions were obtained as oils and later identified as (L,D)-Lac-Phe methyl ester. Similarly, fractions 11-14 were combined, evaporated to dryness to give an oil that was identified by ESI-MS and proton and carbon-NMR as (L,L)-Lac-Phe methyl ester.

TABLE 3
Isolation and purification of (L,D)-Lac-Phe methyl ester and
(L,L)-Lac-Phe methyl ester by flash column chromatography.

| | Hexane | Acetone | |
|---|---|---|---|
| Fractions^{a} | % | % | Composition |
| 1-2 | 95 | 5 | No detectable product |
| 3-4 | 90 | 10 | No detectable product |
| 5-6 | 85 | 15 | No detectable product |
| 7-8 | 80 | 20 | (L,D)-Lac-Phe methyl ester |
| 9-10 | 80 | 20 | (L,D)-and (L,L)-Lac-Phe methyl esters |
| 11-14 | 80 | 20 | (L,L)-Lac-Phe methyl ester |
| 15-18 | 80 | 20 | No detectable product |

^{a}The volume of each fraction was 50 mL.

### Example 3

### Scale-Up Condensation of L-Phenylalanine Methyl Ester Hydrochloride with Ethyl L-Lactate

L-Phenylalanine methyl ester hydrochloride (10.00 g, 0.046 mol), ethyl L-lactate (25.46 g, 0.216 mol), and triethylamine (4.76 g, 0.047 mol) were stirred at ambient temperature for 30 minutes to allow deprotonation of L-phenylalanine methyl ester hydrochloride. Acetic acid (1.40 g, 0.023 mol) was added, and the mixture was heated at 60° C. for 70 h. After work-up according to the procedure in Example 1, 33.4 g of a yellow crude oil was obtained that was subjected to flash column chromatography for isolation of (L,L)- and (L,D)-Lac-Phe methyl esters.

### Example 4

### Large-Scale Isolation and Purification of (L,L)-Lac-Phe Methyl Ester and (L,D)-Lac-Phe Methyl Ester by Flash Column Chromatography

A flash column (length: 380 mm, internal diameter: 40 mL) was packed with silica gel (175 g) using hexane. The crude product (33.4 g) from condensation of L-phenylalanine methyl ester hydrochloride with ethyl L-lactate in acetone (30 mL) was loaded onto the column and the column was eluted with hexane and acetone as shown in Table 4. Based on HPLC analysis, fractions 6-15 were combined and evaporated to dryness to afford 10.20 g of a yellow-orange oil that was shown by HPLC to consists of (L,L)-Lac-Phe methyl ester (74.5%, 7.6 g), (L,D)-Lac-Phe methyl esters (15.4%, 1.57 g), unreacted phenylalanine methyl esters (4.2%, 0.43 g), and an unidentified compound (5.9%). Based on a maximum yield of 11.56 g for Lac-Phe methyl ester, the yield of (L,L)-Lac-Phe methyl ester was 66% and the yield of (L,D)-Lac-Phe methyl ester was 14%. The total recovery of (L,L)- and (L,D)-Lac-Phe corresponded to a combined isolated yield of 79%.

TABLE 4
Large-scale isolation and purification of (L,D)-
Lac-Phe methyl ester and (L,L)-Lac-Phe methyl ester
on 10 g scale by flash column chromatography.

| | Hexane | Acetone | |
|---|---|---|---|
| Fractions^{a} | % | % | Composition |
| 1-2 | 100 | 0 | No detectable product |
| 3-5 | 80 | 20 | No detectable product |
| 6-10 | 80 | 20 | (L,D)-& (L,L)-Lac-Phe methyl esters |
| 11-13 | 75 | 25 | (L,D)-& (L,L)-Lac-Phe methyl esters |
| 14 | 70 | 30 | (L,D)-& (L,L)-Lac-Phe methyl esters |
| 15 | 70 | 30 | (L,L)-Lac-Phe methyl esters, |
| | | | L-Phenylalanine methyl esters |
| 16-18 | 70 | 30 | L-Phenylalanine methyl esters |

^{a}The volume of each fraction was 50 mL.

### Example 5

### Hydrolysis of (L,L)-Lac-Phe Methyl Ester and (L,D)-Lac-Phe Methyl Ester

The crude products (10 g) from condensation of L-phenylalanine methyl ester hydrochloride with ethyl L-lactate that contained a mixture of unreacted L-phenylalanine methyl ester, (L,L)- and (L,D)-Lac-Phe, (L,L)-Lac-Phe methyl ester, and (L,D)-Lac-Phe methyl ester were transferred into a 100 mL flask using acetone (30 g). The mixture was treated with ethanol (20 g), a 50% solution of KOH-water (wt.:wt., 8 g) in acetone (10 g), and the flask was placed in a preheated oil bath at 60° C. and stirred for 45 min. The mixture was allowed to cool down to ambient temperature and was treated with a 50% solution of acetic acid-water (12 mL, v:v) until the solution was acidic (pH=4-5). Acetone was evaporated under reduced pressure (300 torr) at 40° C. and the product was partitioned into ethyl acetate (30 mL) and 5% solution of sodium chloride in water (30 mL). The aqueous layer was removed, and the organic layer was washed with water (3×30 mL), dried over sodium sulfate, and filtered. Ethyl acetate was evaporated under reduced pressure (350-150 torr) at 40-45° C. to afford a dark-yellow oil (9 g) that was subjected to flash column chromatography for isolation and purification of (L,L)- and (L,D)-Lac-Phe.

### Example 6

### Isolation and Purification of (L,L)-Lac-Phe and (L,D)-Lac-Phe by Flash Column Chromatography

A flash column (length: 220 mm, internal diameter: 40 mL) was packed with silica gel (75 g) using hexane. The crude product (9 g) from saponification of (L,D)-Lac-Phe methyl ester and (L,L)-Lac-Phe methyl ester in ethyl acetate (15 mL) was loaded onto the column and the column was eluted with hexane and ethyl acetate as shown in Table 5. Based on HPLC analysis, fractions 7 was evaporated to dryness for MS and NMR analysis; this fraction was later identified as (L,D)-Lac-Phe. Similarly, fractions 10-14 were combined, evaporated to dryness, and were identified by ESI-MS and proton- and carbon-NMR as (L,L)-Lac-Phe.

TABLE 5
Isolation and purification of (L,D)-Lac-Phe and (L,L)-Lac-Phe
by flash column chromatography.

| | Hexane | EtOAc | |
|---|---|---|---|
| Fractions^{a} | % | % | Composition |
| 1-2 | 100 | 0 | No detectable product |
| 3-4 | 75 | 25 | No detectable product |
| 5-6 | 50 | 50 | No detectable product |
| 6 | 50 | 50 | No detectable product |
| 7 | 50 | 50 | (L,D)-Lac-Phe |
| 8 | 50 | 50 | (L,D)-and (L,L)-Lac-Phe |
| 9 | 40 | 60 | (L,D)-and (L,L)-Lac-Phe |
| 10 | 40 | 60 | (L,L)-Lac-Phe |
| 11-12 | 25 | 75 | (L,L)-Lac-Phe |
| 13-14 | 0 | 100 | (L,L)-Lac-Phe |
| 15-16 | 0 | 100 | Trace product detected |

^{a}The volume of each fraction was 125 mL.

It should be appreciated that minor modification to the concentrations of reagent, hydrolysis conditions, and conditions for separation of products, their composition and the ranges expressed herein may be made and still come within the scope and spirit of the present invention.

Having described the invention with reference to particular compositions, theories of effectiveness, and the like, it will be apparent to those of skilled in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

It should be further appreciated that minor dosage and formulation modifications of the composition and the ranges expressed herein may be made and still come within the scope and spirit of the present invention.

It is also to be understood that the formulations and processes described in the specification are simply exemplary embodiments of the inventive concepts defined in the appended claims. Hence, specific conditions and other physical characteristics relating to the embodiments disclosed herein are not to be considered as limiting, unless the claims expressly state otherwise. Where a range of values is provided, it is understood that each intervening value, to the tenth of the unit of the lower limit unless the context clearly dictates otherwise, between the upper and lower limit of that range, and any other stated or intervening value in that stated range, is encompassed within the scope of the present disclosure. The upper and lower limits of these smaller ranges may independently be included in the smaller ranges and are also encompassed within the scope of the present disclosure, subject to any specifically excluded limit in the stated range. Where the stated range includes one or both limits, ranges excluding either or both of those included limits are also included in the scope of the present disclosure. All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, 10) contained within the range. Except in certain examples or where expressly disclosed herein, all numerical values in this specification and the appended claims are understood to be modified by the word “about” when describing the scope of the numerical range.

In this specification and the appended claims, the singular forms “a,” “an” and “the” include plural reference unless the context clearly dictates otherwise. All combinations of method steps or process steps as used herein can be performed in any order, unless otherwise specified or clearly implied to the contrary by the context in which the referenced combination is made.

To the extent that the terms “includes” or “including” or “have” or “having” are used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A” or “B” or both “A” and “B”. When the Applicant intends to indicate “only A or B but not both” then the term “only A or B but not both” or similar structure will be employed. Thus, use of the term “or” herein is the inclusive, and not the exclusive use. Also, to the extent that the terms “in” or “into” are used in the specification or the claims, it is intended to additionally mean “on” or “onto.” In this specification and the appended claims, the singular forms “a,” “an” and “the” include plural reference unless the context clearly dictates otherwise.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

The foregoing description has been presented for the purposes of illustration and description. It is not intended to be an exhaustive list or limit the invention to the precise forms disclosed. It should be appreciated that in some exemplary embodiments, well-known processes, well-known methods, devices, and technologies are not described in detail. Persons of ordinary skill in the art will understand that modifications and variations of the embodiments disclosed can be made within the scope of the present invention in order to achieve substantially similar results.

### REFERENCES

1. Li, Veronica L., Yang He, Kévin Contrepois, Hailan Liu, Joon T. Kim, Amanda L. Wiggenhorn, Julia T. Tanzo, et al. 2022. “An Exercise-Inducible Metabolite That Suppresses Feeding and Obesity.” Nature 606 (7915): 785-90. https://doi.org/10.1038/s41586-022-04828-5
2. Xiao, Shuke, Veronica L. Li, Xuchao Lyu, Xudong Chen, Wei Wei, Fahim Abbasi, Joshua W. Knowles, et al. 2023. “Lac-Phe Mediates the Anti-Obesity Effect of Metformin.” bioRxiv. https://doi.org/10.1101/2023.11.02.565321.
3. Wu, Jing, Jingrong Gao, Junjie Lin, Chun Cui, Laihao Li, Shan He, and Charles Brennan. 2022. “Preparation and Taste Characteristics of Kokumi N-Lactoyl Phenylalanine in the Presence of Phenylalanine and Lactate.” Journal of Agricultural and Food Chemistry 70 (17): 5396-5407. https://doi.org/10.1021/acs.jafc.2c00530.
4. Gooßen, Lukas, Dominik Ohlmann, and Paul Lange. 2009. “The Thermal Amidation of Carboxylic Acids Revisited.” Synthesis 2009 (01): 160-64. https://doi.org/10.1055/s-0028-1083277.
5. Taussat, Armand, Renata Marcia de Figueiredo, and Jean-Marc Campagne. 2023. “Direct Catalytic Amidations from Carboxylic Acid and Ester Derivatives: A Review.” Catalysts 13 (2): 366. https://doi.org/10.3390/catal13020366.
6. Sharley, Daniel D. Sanz, and Jonathan M. J. Williams. 2017. “Acetic Acid as a Catalyst for the N-Acylation of Amines Using Esters as the Acyl Source.” Chemical Communications 53 (12): 2020-23. https://doi.org/10.1039/C6CC09023K.

1. A process for obtaining (L,L)-N-Lactoyl-Phenylalanine ester [(L,L)-Lac-Phe ester] comprising the steps of:
treating a hydrochloride salt of L-phenylalanine ester with a tertiary amine in a solvent to obtain a reaction mixture;
adding acetic acid to the reaction mixture; and
heating the reaction mixture to about 45-95° C. to obtain (L,L)-Lac-Phe ester and (L,D)-Lac-Phe ester.

2. The process of claim 1, wherein the ester of L-phenylalanine hydrochloride is chosen from the group consisting of methyl, ethyl, n-propyl, or isopropyl.

3. The process of claim 1, wherein the solvent is L-lactic acid ester and wherein the ester of L-lactic acid is chosen from the group consisting of methyl, ethyl, n-propyl, or isopropyl.

4. The process of claim 1, wherein the tertiary amine is chosen from the group consisting of triethylamine, tripropylamine, or tributylamine.

5. The process of claim 1, wherein one equivalent of L-phenylalanine ester is treated with four equivalents of solvent, one equivalent of triethylamine, and 0.1 to 1 equivalent of acetic acid.

6. The process of claim 1, wherein the reaction mixture is filtered to remove the hydrochloride salt of amine using an organic solvent.

7. The process of claim 6, wherein the organic solvent is evaporated under reduced pressure to obtain a crude mixture of (L,L)-Lac-Phe ester and (L,D)-Lac-Phe ester.

8. The process of claim 1, wherein the reaction mixture comprises about 60 to 77% (L,L)-Lac-Phe ester, about 7 to 16% (L,D)-Lac-Phe ester, and about 4 to 22% unreacted L-phenylalanine ester.

9. The process of claim 1, wherein (L,L)-Lac-Phe ester is obtained in about 60 to 82% diastereomeric excess.

10. The process of claim 1, further comprising separation and isolation of (L,L)-Lac-Phe ester from (L,D)-Lac-Phe ester by loading the mixture onto a silica gel column and eluting the column with hexane and acetone.

11. The process of claim 1, wherein saponification is carried out in an alcohol and a base such as aqueous sodium hydroxide or potassium hydroxide at about 50 to 60° C.

12. The process of claim 1, further comprising purifying (L,L)-Lac-Phe and (L,D)-Lac-Phe by loading the mixture onto a silica gel column and eluting the column with hexane and ethyl acetate.

13. The process of claim 1, wherein the composition of hexane is in the range of 100-0% and ethyl acetate is in the range of 0-100%.

14. A process for obtaining (L,D)-Lac-Phe ester comprising the steps of:
treating a hydrochloride salt of D-phenylalanine ester with a tertiary amine in L-lactic acid ester to obtain a reaction mixture;
adding an organic solvent and heating the reaction mixture to a temperature ranging from about 45-95° C. to obtain (L,D)-Lac-Phe ester and (L,L)-Lac-Phe ester.

15. The process of claim 14, wherein the ester of D-phenylalanine hydrochloride is chosen from the group consisting of methyl, ethyl, n-propyl, or isopropyl.

16. The process of claim 14, wherein the ester of L-lactic acid is chosen from the group consisting of methyl, ethyl, n-propyl, or isopropyl.

17. The process of claim 14, wherein the tertiary amine is chosen from the group consisting of triethylamine, tripropylamine, or tributylamine.

18. The process of claim 14, wherein 1 equivalent of D-phenylalanine ester is treated with 4 equivalent of L-lactic acid ester, 1 equivalent of triethylamine, and between 0.1 to 1 equivalent of acetic acid.

19. The process of claim 14, wherein the reaction mixture is filtered to remove the hydrochloride salt of amine using an organic solvent.

20. The process of claim 19, wherein the organic solvent is evaporated under reduced pressure to obtain a crude mixture of (L,D)-Lac-Phe ester and (L,L)-Lac-Phe ester.

21. The process of claim 14, wherein the relative composition of the product is: (L,D)-Lac-Phe ester in an amount ranging from about 60 to 77% by weight, (L,L)-Lac-Phe ester in an amount ranging from about 7 to 16% by weight, and unreacted L-phenylalanine ester in an amount ranging from about 4 to 22% by weight.

22. The process of claim 14, wherein (L,D)-Lac-Phe ester is obtained in about 60-82% diastereomeric excess.

23. The process of claim 14, wherein the crude product without purification is directly subjected to alkaline hydrolysis to obtain (L,D)-Lac-Phe and (L,L)-Lac-Phe.

24. The process of claim 23, wherein saponification is carried out in an alcohol at a temperature of about 50 to 60° C.

25. A process for synthesis of (L,L)-N-Lactoyl-Phenylalanine ester [(L,L)-Lac-Phe ester comprising the steps:
a. deprotonating L-phenylalanine methyl ester hydrochloride with triethylamine at ambient temperature using ethyl L-lactate as solvent to create a reaction mixture;
b. treating the reaction mixture with an organic solvent;
c. heating the reaction mixture to yield (L,L)-Lac-Phe methyl ester and (L,D)-Lac-Phe methyl ester; and
d. subjecting the reaction mixture to alkaline hydrolysis to yield (L,L)-Lac-Phe and (L,D)-Lac-Phe.
